(19) United States
(12) Patent Application Publication
(10) Pub. No.: US 2015/0196214 A1
(43) Pub. Date: Jul. 16, 2015

(54) MEDICAL DEVICES FOR MAPPING CARDIAC TISSUE

(71) Applicant: BOSTON SCIENTIFIC SCIMED, INC., MAPLE GROVE, MN (US)

(72) Inventors: ALLAN C. SHUROS, ST. PAUL, MN (US); PRAMODSINGH HIRASINGH THAKUR, WOODBURY, MN (US); SHIBAJI SHOME, ARDEN HILLS, MN (US); BARUN MASKARA, BLAINE, MN (US); SHANTHA ARCOT-KRISHNAMURTHY, RENTON, WA (US); JACOB I. LAUGHNER, ST. PAUL, MN (US)

(73) Assignee: BOSTON SCIENTIFIC SCIMED, INC., MAPLE GROVE, MN (US)

(21) Appl. No.: 14/594,642

(22) Filed: Jan. 12, 2015

Related U.S. Application Data
(60) Provisional application No. 61/926,727, filed on Jan. 13, 2014.

(51) Int. Cl. A61B 5/042; A61B 18/14; A61B 5/0452; A61B 5/00; A61B 5/044; A61B 5/04

(52) U.S. Cl. CPC A61B 5/0422; A61B 5/044; A61B 5/04012; A61B 5/0452; A61B 5/4836; A61B 18/1492; A61B 2018/00357

(57) ABSTRACT

Medical devices and methods for making and using medical devices are disclosed. An example medical device may include a catheter shaft with a plurality of electrodes coupled thereto and a processor coupled to the catheter shaft. The processor may be capable of collecting a set of signals from the plurality of electrodes, characterizing the set of signals, generating a visual representation of the set of signals and refining the visual representation.

### CROSS-REFERENCE TO RELATED APPLICATIONS

This application claims priority under 35 U.S.C. §119 to U.S. Provisional Application Ser. No. 61/926,727, filed Jan. 13, 2014, the entirety of which is incorporated herein by reference.

### TECHNICAL FIELD

The present disclosure pertains to medical devices, and methods for manufacturing medical devices. More particularly, the present disclosure pertains to medical devices and methods for mapping and/or ablating cardiac tissue.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for medical devices. An example medical device is disclosed. The medical device comprises:

a catheter shaft with a plurality of electrodes coupled thereto; and

a processor coupled to the catheter shaft, wherein the processor is capable of:
collecting a set of signals from at least some of the plurality of electrodes;
generating a data set from the set of signals;
performing a data reduction process on the data set; and
generating a visual representation of the data set.

Additionally or alternatively to any of the examples above, the collecting the set of signals includes sensing a change in electrical potential by any one of the plurality of electrodes.

Additionally or alternatively to any of the examples above, further comprising identifying a threshold value corresponding to a minimum change in electrical potential by any one of the plurality of electrodes; and wherein collecting the set of signals includes collecting only those signals that are above the threshold value.

Additionally or alternatively to any of the examples above, wherein collecting the set of signals includes determining an activation time at one or more of the plurality of electrodes.

Additionally or alternatively to any of the examples above, wherein determining the activation time includes identifying a fiducial point corresponding to a change in electrical potential and determining a time latency between a reference point and the fiducial point.

Additionally or alternatively to any of the examples above, wherein performing the data reduction process on the data set includes assigning a rank category to the activation times.

Additionally or alternatively to any of the examples above, wherein each rank category corresponds to a discrete activation time interval.

Additionally or alternatively to any of the examples above, wherein generating a visual representation includes creating an activation map.

Additionally or alternatively to any of the examples above, wherein the activation map includes a grid displaying a plurality of activation times.

Additionally or alternatively to any of the examples above, wherein the activation map includes a grid displaying a plurality of rank categories.

Additionally or alternatively to any of the examples above, wherein the activation map includes activation times from only some of the plurality of electrodes and includes one or more missing activation times.

Additionally or alternatively to any of the examples above, further comprising assigning activation times to the one or more missing activation times.

Additionally or alternatively to any of the examples above, wherein assigning activation times to the one or more missing activation times includes adopting the value of at least one activation time.

Additionally or alternatively to any of the examples above, wherein the activation map includes rank categories from only some of the plurality of electrodes and includes one or more missing rank categories.

Additionally or alternatively to any of the examples above, further comprising assigning rank categories to the one or more missing rank categories.

Additionally or alternatively to any of the examples above, wherein assigning rank categories to the one or more missing rank categories includes adopting the value of at least one rank category.

Additionally or alternatively to any of the examples above, wherein the activation map further comprises a plurality of color indicators.

Additionally or alternatively to any of the examples above, wherein the color indicators represent activation times.

Additionally or alternatively to any of the examples above, wherein the color indicators represent rank categories.

Additionally or alternatively to any of the examples above, wherein the visual representation includes a key correlating the color indicators to the activation times.

Additionally or alternatively to any of the examples above, wherein the visual representation includes a key correlating the color indicators to the rank categories.

Additionally or alternatively to any of the examples above, wherein the visual representation is displayed on a display.

A method for delivering a medical mapping device is disclosed. The method comprises:

delivering the medical mapping device of any one of claims 1-22 into the heart of a patient.

A medical device for mapping a cardiac chamber is disclosed. The medical device comprises:

a catheter shaft with a plurality of electrodes coupled thereto;

a processor, wherein the processor is capable of:
collecting a set of signals from at least some of the plurality of electrodes;
generating a data set from at least one of the set of signals, wherein the data set includes at least one known data point and one or more unknown data points;
performing a data reduction process on the known data point; and
assigning a value to at least one of the unknown data points.

Additionally or alternatively to any of the examples above, wherein collecting the set of signals further includes sensing a change in electrical potential by any one of the plurality of electrodes.

Additionally or alternatively to any of the examples above, further comprising identifying a threshold value corresponding to a minimum change in electrical potential by any one of the plurality of electrodes; and wherein collecting the set of signals includes collecting only those signals that are above the threshold value.

Additionally or alternatively to any of the examples above, wherein generating the set of signals includes determining an activation time at one or more of the plurality of electrodes with the at least one known data point.

Additionally or alternatively to any of the examples above, wherein determining the activation time includes identifying a fiducial point corresponding to a change in electrical potential and determining a time latency between a reference point and the fiducial point.

Additionally or alternatively to any of the examples above, wherein performing the data reduction process on the data set includes assigning a rank category to the activation times.

Additionally or alternatively to any of the examples above, wherein each rank category corresponds to a discrete activation time interval.

Additionally or alternatively to any of the examples above, wherein assigning a value to at least one of the unknown data points includes adopting the value of at least one activation time.

Additionally or alternatively to any of the examples above, wherein assigning a value to at least one of the unknown data points includes adopting the value of at least one rank category.

Additionally or alternatively to any of the examples above, further comprising generating a visual display.

Additionally or alternatively to any of the examples above, wherein the visual display includes an activation map.

Additionally or alternatively to any of the examples above, wherein the activation map includes activation times.

Additionally or alternatively to any of the examples above, wherein the activation map includes rank categories.

Additionally or alternatively to any of the examples above, wherein the activation map further comprises a plurality of color indicators.

Additionally or alternatively to any of the examples above, wherein the color indicators represent activation times.

Additionally or alternatively to any of the examples above, wherein the color indicators represent rank categories.

Additionally or alternatively to any of the examples above, wherein the visual display includes a key correlating the color indicators to the activation times.

Additionally or alternatively to any of the examples above, wherein the display includes a key correlating the color indicators to rank categories.

A method of mapping the electrical activity of the heart is disclosed. The method comprises:

advancing a catheter shaft with a plurality of electrodes coupled thereto to a chamber of the heart, wherein the catheter shaft is coupled to a processor, and wherein the processor is configured to:
collect a set of signals from at least some of the plurality of electrodes;
generate a data set from the set of signals;
perform a data reduction process on the data set; and
generate a visual representation of the data set.

Additionally or alternatively to any of the examples above, wherein collecting the set of signals includes sensing a change in electrical potential by any one of the plurality of electrodes.

Additionally or alternatively to any of the examples above, further comprising identifying a threshold value corresponding to a minimum change in electrical potential by any one of the plurality of electrodes; and wherein collecting the set of signals includes collecting only those signals that are above the threshold value.

Additionally or alternatively to any of the examples above, wherein collecting the set of signals includes determining an activation time at one or more of the plurality of electrodes.

Additionally or alternatively to any of the examples above, wherein determining the activation time includes identifying a fiducial point corresponding to a change in electrical potential and determining a time latency between a reference point and the fiducial point.

Additionally or alternatively to any of the examples above, wherein performing the data reduction process on the data set includes assigning a rank category to the activation times.

Additionally or alternatively to any of the examples above, wherein each rank category corresponds to a discrete activation time interval.

Additionally or alternatively to any of the examples above, wherein generating a visual representation includes creating an activation map.

Additionally or alternatively to any of the examples above, wherein the activation map includes a grid displaying a plurality of activation times.

Additionally or alternatively to any of the examples above, wherein the activation map includes a grid displaying a plurality of rank categories.

Additionally or alternatively to any of the examples above, wherein the activation map includes activation times from only some of the plurality of electrodes and includes one or more missing activation times.

Additionally or alternatively to any of the examples above, further comprising assigning activation times to the one or more missing activation times.

Additionally or alternatively to any of the examples above, wherein assigning activation times to the one or more missing activation times includes adopting the value of at least one activation time.

Additionally or alternatively to any of the examples above, wherein the activation map includes rank categories from only some of the plurality of electrodes and includes one or more missing rank categories.

Additionally or alternatively to any of the examples above, further comprising assigning rank categories to the one or more missing rank categories.

Additionally or alternatively to any of the examples above, wherein assigning rank categories to the one or more missing rank categories includes adopting the value of at least one rank category.

Additionally or alternatively to any of the examples above, wherein the activation map further comprises a plurality of color indicators.

Additionally or alternatively to any of the examples above, wherein the color indicators represent activation times.

Additionally or alternatively to any of the examples above, wherein the color indicators represent rank categories.

Additionally or alternatively to any of the examples above, wherein the visual representation includes a key correlating the color indicators to the activation times.

Additionally or alternatively to any of the examples above, wherein the visual representation includes a key correlating the color indicators to the rank categories.

Additionally or alternatively to any of the examples above, wherein the visual representation is displayed on a display.

A medical device for mapping a cardiac chamber is disclosed. The medical device comprises:

a catheter shaft with a plurality of electrodes coupled thereto;

a processor, wherein the processor is capable of:
collecting a set of signals from at least some of the plurality of electrodes;
generating a data set from at least one of the set of signals, wherein the data set includes at least one known activation time and one or more unknown activation times;
performing a data reduction process on the at least one known activation time; and
assigning a value to at least one of the unknown activation times.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example catheter system for accessing a targeted tissue region in the body for diagnostic and therapeutic purposes.

FIG. 2 is a schematic view of an example mapping catheter having a basket functional element carrying structure for use in association with the system of FIG. 1.

FIG. 3 is a schematic view of an example functional element including a plurality of mapping electrodes.

FIG. 4 illustrates an example activation map.

FIG. 5 is an illustration of example electrograms displaying fiducial points, time activation identifiers and discrete time intervals.

FIG. 6 illustrates an example “data reduced” activation map.

FIG. 7 illustrates an example activation map where missing data is filled in.

FIG. 8 illustrates an example activation map and key utilizing a pattern and/or texture to represent unique rank categories.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Mapping the electrophysiology of heart rhythm disorders often involves the introduction of a constellation catheter or other mapping/sensing device having a plurality of sensors into a cardiac chamber. The sensors detect the electric activity of the heart at sensor locations. It may be desirable to have the electric activity processed into electrogram signals that accurately represent cellular excitation through cardiac tissue relative to the sensor locations. A processing system may then analyze and output the signal to a display device. Further, the processing system may output the signal as an activation map. The physician may use the activation map to perform a diagnostic procedure.

However, in some cases the sensing electrodes may fail to accurately detect the electrical activity of heart. The failure of the electrodes to detect a signal may limit and/or reduce the ability of the processing system to accurately display information used for diagnostic procedures. For example, an activation map may be generated that contains missing information and/or inaccurate visual representations. Therefore, it may be desirable to replace poor or non-existent electrical signal information with information that is believed to be accurate. In some instances, interpolation may be used to replace poor/missing data. Standard interpolation methods may have limitations due to both the temporal nature of the activation signals and the three-dimensional spatial configuration of sensing electrodes located in an anatomical region. The methods and systems disclosed herein are designed to overcome at least some of the limitations of standard interpolation methods used to interpolate poor or non-existent activation signals. For example, some of the methods disclosed herein may utilize data reduction processes in order to simplify activation maps or otherwise replace/fill in poor or non-existent data. Other methods and medical devices are also disclosed.

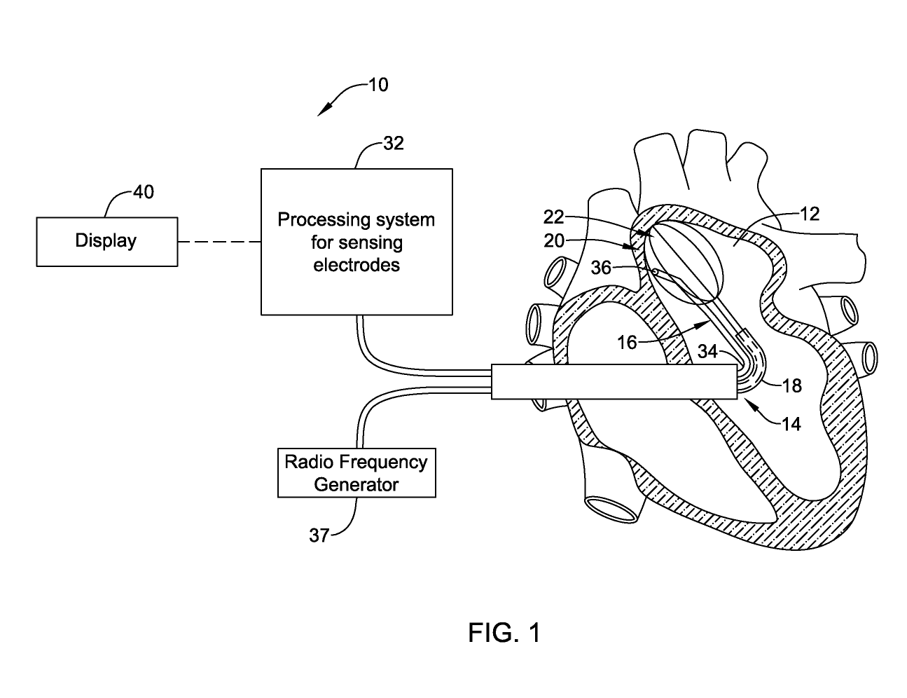

FIG. 1 is a schematic view of a system 10 for accessing a targeted tissue region in the body for diagnostic and/or therapeutic purposes. FIG. 1 generally shows the system 10 deployed in the left atrium of the heart. Alternatively, system 10 can be deployed in other regions of the heart, such as the left ventricle, right atrium, or right ventricle. While the illustrated embodiment shows the system 10 being used for ablating myocardial tissue, the system 10 (and the methods described herein) may alternatively be configured for use in other tissue ablation applications, such as procedures for ablating tissue in the prostrate, brain, gall bladder, uterus, nerves, blood vessels and other regions of the body, including in systems that are not necessarily catheter-based.

The system 10 includes a mapping probe 14 and an ablation probe 16. Each probe 14/16 may be separately introduced into the selected heart region 12 through a vein or artery (e.g., the femoral vein or artery) using a suitable percutaneous access technique. Alternatively, the mapping probe 14 and ablation probe 16 can be assembled in an integrated structure for simultaneous introduction and deployment in the heart region 12.

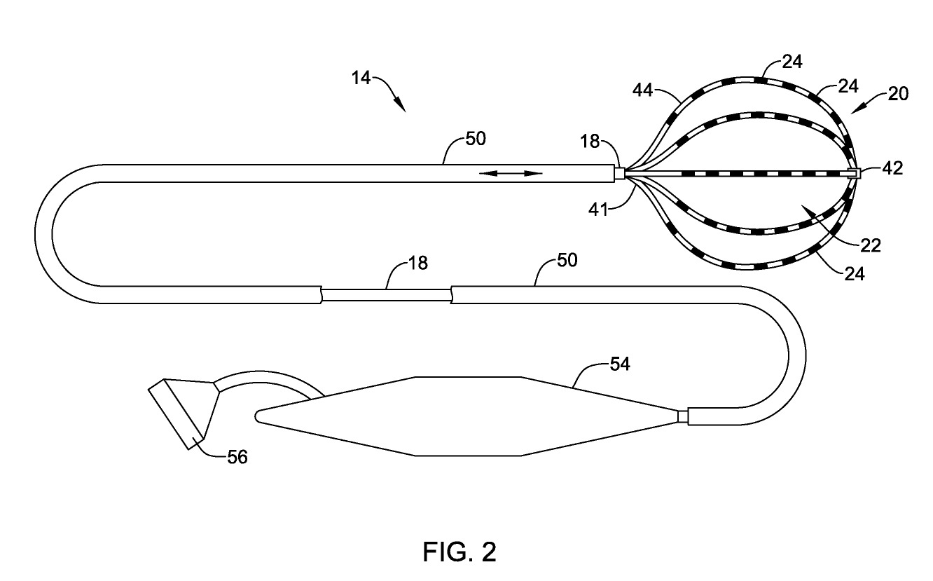

The mapping probe 14 may have a flexible catheter body 18. The distal end of the catheter body 18 carries a three-dimensional multiple electrode structure 20. In the illustrated embodiment, the structure 20 takes the form of a basket defining an open interior space 22 (see FIG. 2), although other multiple electrode structures could be used. The multiple electrode structure 20 carries a plurality of mapping electrodes 24 (not explicitly shown on FIG. 1, but shown on FIG. 2) each having an electrode location on structure 20 and a conductive member. Each electrode 24 may be configured to sense intrinsic physiological activity in the anatomical region. In some embodiments, the electrodes 24 may be configured to detect activation signals of the intrinsic physiological activity within the anatomical structure (e.g., the activation times of cardiac activity).

The electrodes 24 are electrically coupled to a processing system 32. A signal wire (not shown) may be electrically coupled to each electrode 24 on the basket structure 20. The wires may extend through the body 18 of the probe 14 and electrically couple each electrode 24 to an input of the processing system 32. The electrodes 24 sense electrical activity in the anatomical region, e.g., myocardial tissue. The sensed activity (e.g., activation signals) may be processed by the processing system 32 to assist the physician by generating an anatomical map (e.g., a vector field map) to identify the site or sites within the heart appropriate for a diagnostic and/or treatment procedure, e.g. an ablation procedure. For example, the processing system 32 may identify a near-field signal component (e.g., activation signals originating from cellular tissue adjacent to the mapping electrode 24) or from an obstructive far-field signal component (e.g., activation signals originating from non-adjacent tissue). For example, the near-field signal component may include activation signals originating from atrial myocardial tissue whereas the far-field signal component may include activation signals originating from ventricular myocardial tissue. The near-field activation signal component may be further analyzed to find the presence of a pathology and to determine a location suitable for ablation for treatment of the pathology (e.g., ablation therapy).

The processing system 32 may include dedicated circuitry (e.g., discrete logic elements and one or more microcontrollers; application-specific integrated circuits (ASICs); or specially configured programmable devices, such as, for example, programmable logic devices (PLDs) or field programmable gate arrays (FPGAs)) for receiving and/or processing the acquired activation signals. In some embodiments, the processing system 32 includes a general purpose microprocessor and/or a specialized microprocessor (e.g., a digital signal processor, or DSP, which may be optimized for processing activation signals) that executes instructions to receive, analyze and display information associated with the received activation signals. In such implementations, the processing system 32 can include program instructions, which when executed, perform part of the signal processing. Program instructions can include, for example, firmware, microcode or application code that is executed by microprocessors or microcontrollers. The above-mentioned implementations are merely exemplary, and the reader will appreciate that the processing system 32 can take any suitable form.

In some embodiments, the processing system 32 may be configured to measure the electrical activity in the myocardial tissue adjacent to the electrodes 24. For example, in some embodiments, the processing system 32 is configured to detect electrical activity associated with a dominant rotor or divergent activation pattern in the anatomical feature being mapped. For example, dominant rotors and/or divergent activation patterns may have a role in the initiation and maintenance of atrial fibrillation, and ablation of the rotor path, rotor core, and/or divergent foci may be effective in terminating the atrial fibrillation. In either situation, the processing system 32 processes the sensed activation signals to generate a display of relevant characteristic, such as an isochronal map, activation time map, action potential duration (APD) map, a vector field map, a contour map, a reliability map, an electrogram, a cardiac action potential and the like. The relevant characteristics may be used by the physician to identify a site suitable for ablation therapy.

The ablation probe 16 includes a flexible catheter body 34 that carries one or more ablation electrodes 36. The one or more ablation electrodes 36 are electrically connected to a radio frequency (RF) generator 37 that is configured to deliver ablation energy to the one or more ablation electrodes 36. The ablation probe 16 may be movable with respect to the anatomical feature to be treated, as well as the structure 20. The ablation probe 16 may be positionable between or adjacent to electrodes 24 of the structure 20 as the one or more ablation electrodes 36 are positioned with respect to the tissue to be treated.

The processing system 32 may output data to a suitable output or display device 40, which may display relevant information for a clinician. In the illustrated embodiment, device 40 is a CRT, LED, or other type of display, or a printer. Device 40 presents the relevant characteristics in a format most useful to the physician. In addition, processing system 32 may generate position-identifying output for display on device 40 that aids the physician in guiding ablation electrode(s) 36 into contact with tissue at the site identified for ablation.

FIG. 2 illustrates mapping catheter 14 and shows electrodes 24 at the distal end suitable for use in the system 10 shown in FIG. 1. Mapping catheter 14 may have a flexible catheter body 18, the distal end of which may carry three dimensional structure 20 with mapping electrodes or sensors 24. Mapping electrodes 24 may sense electrical activity (e.g., activation signals) in the myocardial tissue. The sensed activity may be processed by the processing system 32 to assist the physician in identifying the site or sites having a heart rhythm disorder or other myocardial pathology via generated and displayed relevant characteristics. This information can then be used to determine an appropriate location for applying appropriate therapy, such as ablation, to the identified sites, and to navigate the one or more ablation electrodes 36 to the identified sites.

The illustrated three-dimensional structure 20 comprises a base member 41 and an end cap 42 between which flexible splines 44 generally extend in a circumferentially spaced relationship. As discussed herein, the three dimensional structure 20 may take the form of a basket defining an open interior space 22. In some embodiments, the splines 44 are made of a resilient inert material, such as Nitinol, other metals, silicone rubber, suitable polymers, or the like and are connected between the base member 41 and the end cap 42 in a resilient, pretensioned condition, to bend and conform to the tissue surface they contact. In the illustrated embodiment, eight splines 44 form the three dimensional structure 20. Additional or fewer splines 44 could be used in other embodiments. As illustrated, each spline 44 carries eight mapping electrodes 24. Additional or fewer mapping electrodes 24 could be disposed on each spline 44 in other embodiments of the three dimensional structure 20. In the illustrated embodiment, the three dimensional structure 20 is relatively small (e.g., 40 mm or less in diameter). In alternative embodiments, the three dimensional structure 20 is even smaller or larger (e.g., 40 mm in diameter or greater).

A slidable sheath 50 may be movable along the major axis of the catheter body 18. Moving the sheath 50 distally relative to catheter body 18 may cause sheath 50 to move over the three dimensional structure 20, thereby collapsing the structure 20 into a compact, low profile condition suitable for introduction into and/or removal from an interior space of an anatomical structure, such as, for example, the heart. In contrast, moving the sheath 50 proximally relative to the catheter body may expose the three dimensional structure 20, allowing the structure 20 to elastically expand and assume the pretensed position illustrated in FIG. 2.

A signal wire (not shown) may be electrically coupled to each mapping electrode 24. The wires may extend through the body 18 of the mapping catheter 20 (or otherwise through and/or along the body 18) into a handle 54, in which they are coupled to an external connector 56, which may be a multiple pin connector. The connector 56 electrically couples the mapping electrodes 24 to the processing system 32. These are just examples. Some addition details regarding these and other example mapping systems and methods for processing signals generated by the mapping catheter can be found in U.S. Pat. Nos. 6,070,094, 6,233,491, and 6,735,465, the disclosures of which are hereby expressly incorporated herein by reference.

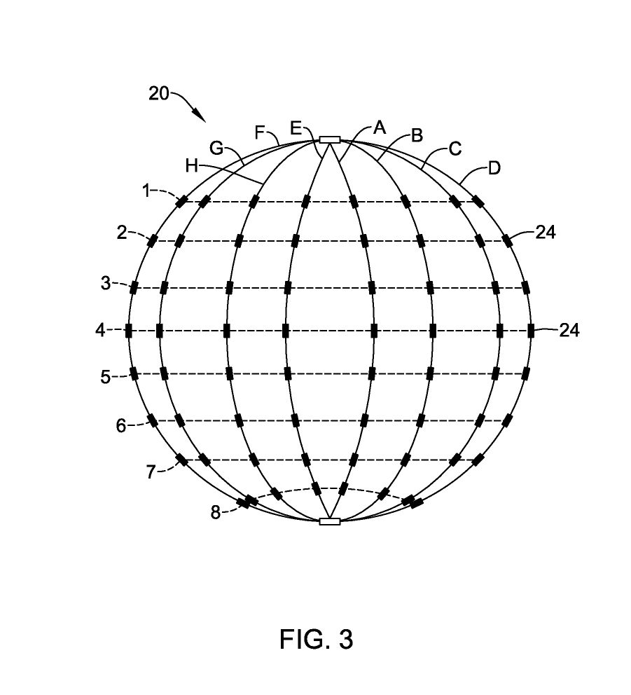

To illustrate the operation of the system 10, FIG. 3 is a schematic side view of an embodiment of the basket structure 20 including a plurality of mapping electrodes 24. In the illustrated embodiment, the basket structure includes 64 mapping electrodes 24. The mapping electrodes 24 are disposed in groups of eight electrodes (labeled 1, 2, 3, 4, 5, 6, 7, and 8) on each of eight splines (labeled A, B, C, D, E, F, G, and H). While an arrangement of sixty-four mapping electrodes 24 is shown disposed on a basket structure 20, the mapping electrodes 24 may alternatively be arranged in different numbers (more or fewer splines and/or electrodes), on different structures, and/or in different positions. In addition, multiple basket structures can be deployed in the same or different anatomical structures to simultaneously obtain signals from different anatomical structures.

After the basket structure 20 is positioned adjacent to the anatomical structure to be treated (e.g. left atrium, left ventricle, right atrium, or right ventricle of the heart), the processing system 32 is configured to record the activation signals from each electrode 24 channel related to physiological activity of the anatomical structure (e.g., the electrodes 24 measure electrical activation signals associated with the physiology of the anatomical structure). The activation signals of physiological activity may be sensed in response to intrinsic physiological activity or based on a predetermined pacing protocol instituted by at least one of the plurality of electrodes 24.

The arrangement, size, spacing and location of electrodes along a constellation catheter or other mapping/sensing device, in combination with the specific geometry of the targeted anatomical structure, may contribute to the ability (or inability) of electrodes 24 to sense, measure, collect and transmit electrical activity of cellular tissue. As stated, because splines 44 of a mapping catheter, constellation catheter or other similar sensing device are bendable, they may conform to a specific anatomical region in a variety of shapes and/or configurations. Further, at any given position in the anatomical region, the electrode basket structure 20 may be manipulated such that one or more splines 44 may not contact adjacent physiological tissue. For example, splines 44 may twist, bend or lie atop one another, thereby separating splines 44 from nearby physiological tissue. Additionally, because electrodes 24 are disposed on one or more of splines 44, they also may not maintain contact with adjacent physiological tissue.

In addition to that stated above, electrodes 24 may not be in contact with adjacent physiological tissue for other reasons. For example, manipulation of mapping catheter 14 may result in movement of electrodes 24, thereby creating poor electrode-to-tissue contact. Further, electrodes 24 may be positioned adjacent fibrous, dead or functionally refractory tissue. Electrodes 24 positioned adjacent fibrous, dead or functionally refractory tissue may not be able to sense changes in electrical potential because fibrous, dead or functionally refractory tissue may be incapable of depolarizing and/or responding to changes in electrical potential. Finally, far-field ventricular events and electrical line noise may distort measurement of tissue activity.

However, electrodes 24 that contact healthy, responsive cellular tissue may sense a change in the voltage potential of a propagating cellular activation wavefront. The change in voltage potential of a cellular tissue may be sensed, collected and displayed as an electrogram. An electrogram may be a visual representation of the change in voltage potential of the cellular tissue over time. Additionally, it may be desirable to define a specific characteristic of an electrogram as a “fiducial” point of the electrical signal. For purposes of this disclosure, a fiducial point may be understood as a characteristic of an electrogram that can be utilized as an identifying characteristic of cellular activation. Fiducial points may correspond to the peak amplitude, change in slope, and/or deflection of the electrical signal. It is contemplated that fiducial points may include other characteristics of an electrogram. Further, fiducial points may be identified manually by a clinician and/or automatically by processing system 32.

An electrogram representing a change in voltage potential over time may be defined as visually displaying the electrical signal in the “time domain.” However, it is generally understood that any electrical signal has a corollary representation in the frequency domain. Transforms (e.g. Fourier) may be utilized to transform signals between the time (spatial) domain and frequency domain, as desired. It is contemplated that at least some embodiments disclosed herein may be equally applied to signals in both the time and frequency domain. Further, it is contemplated that at least some embodiments disclosed herein may be equally applied to the derivatives of any signal, in both the time and frequency domain. Additionally, it is contemplated that at least some embodiments disclosed herein may be equally applied to the transform (e.g., Hilbert, etc.) of any signal in both the time and frequency domain.

As suggested herein, fiducial points may be used to identify the “activation,” or firing, of cellular tissue. Therefore, processing system 32 may calculate the “activation time” of cellular firing by calculating the time a fiducial point is identified as compared to a reference time point. The reference time point may include the time at which a cellular activation wavefront passes a reference electrode. Processing system 32 may identify the activation times of cellular tissue excitation as a cellular activation wavefront moves underneath multiple electrodes 24. For example, processing system 32 may identify the fiducial points on an example electrogram generated by sensing electrical activity with an electrode (e.g., an example electrode that for simplicity purposes will be termed “electrode E1”). In this example, the example electrode E1 may be positioned at a point in space (e.g., within a cardiac chamber) adjacent to one or more additional electrodes (e.g., for simplicity purposes, example electrode E1 may be positioned adjacent to another example electrode that will be termed example electrode E2). Processing system 32 may calculate the activation times of example electrodes E1 and E2 as a cellular wavefront moves underneath electrodes E1 and E2, respectively. Further, a cellular activation wavefront that passes underneath example electrode E1 at a time before it passes under example electrode E2 may be sensed by electrode E1 prior to being sensed by electrode E2. In other words, electrode E2 may sense the wavefront at a later point in time as compared to E1. The activation time delay between the firing of cells underneath E1 and cells underneath E2 may be referred to as the “latency” between E1 and E2.

Therefore, it may be appreciated that as a cellular wavefront passes underneath basket electrode structure 20, processing system 32 may identify the activation time(s) of one or more of electrodes 24 (relative to a reference time point). In some embodiments, it may be desirable to define activation times on a subset of signals. For example, it may be desirable to identify and compare a subset of signals that reach a threshold change in voltage potential. It is further contemplated that other characterizations and/or signal properties may be used to identify the specific signals used for comparison. Further, it is possible that a fiducial point may not be sensed by some electrodes. As stated, some electrodes may not sense cellular activation because adjacent tissue is incapable of depolarizing (e.g. adjacent fibrous, dead or functionally refractory tissue) and/or the electrode has poor electrode-to-tissue contact.

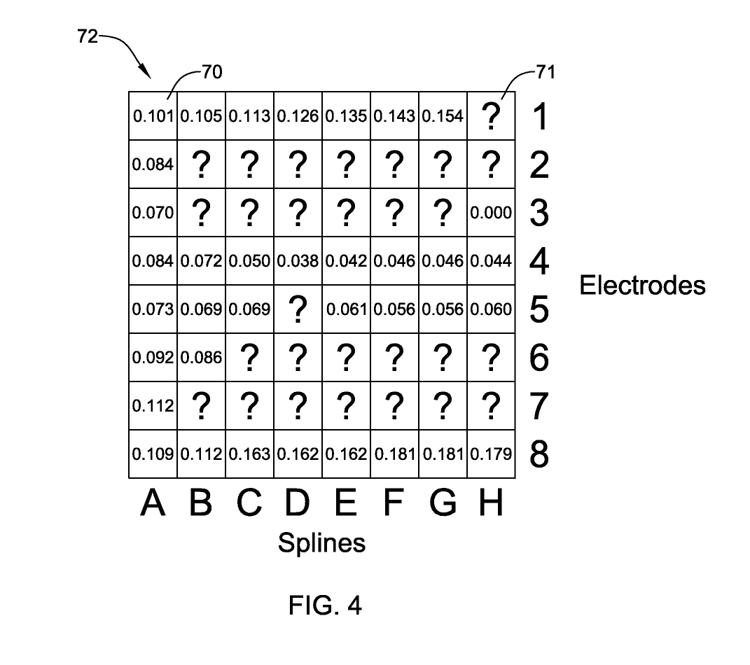

Once a subset of electrical signals has been identified, it may be desirable to compare and categorize the activation times of cellular tissue across the subset of signals. FIG. 4 illustrates an example activation map 72 showing activation times sensed by electrodes 24. In this example, activation map 72 takes the form of a grid that is designed to display collected activation times for all 64 electrodes 24 of multiple electrode structure 20. For example, a space 70 on map 72 representing electrode 1 on spline A displays an activation time of 0.101 ms. Conversely, one or more spaces like a space 71 representing electrode 1 on spline H may display a “?.” The “?” may indicate that the particular electrode corresponding to that location on the multiple electrode structure 20 cannot sense an activation time. Therefore, the “?” may represent missing signal data.

It may be desirable to simplify activation map 72. In order to simplify map 72, a number of data reduction steps may be utilized. The data reduction steps may include using one or more processes to substitute the raw activation times displayed on map 72 with a representative number, color, texture, or other visual indicator that simplifies map 72. In addition, one or more additional processes may be utilized in order to fill in missing data (e.g., data displayed as a “?”) on map 72.

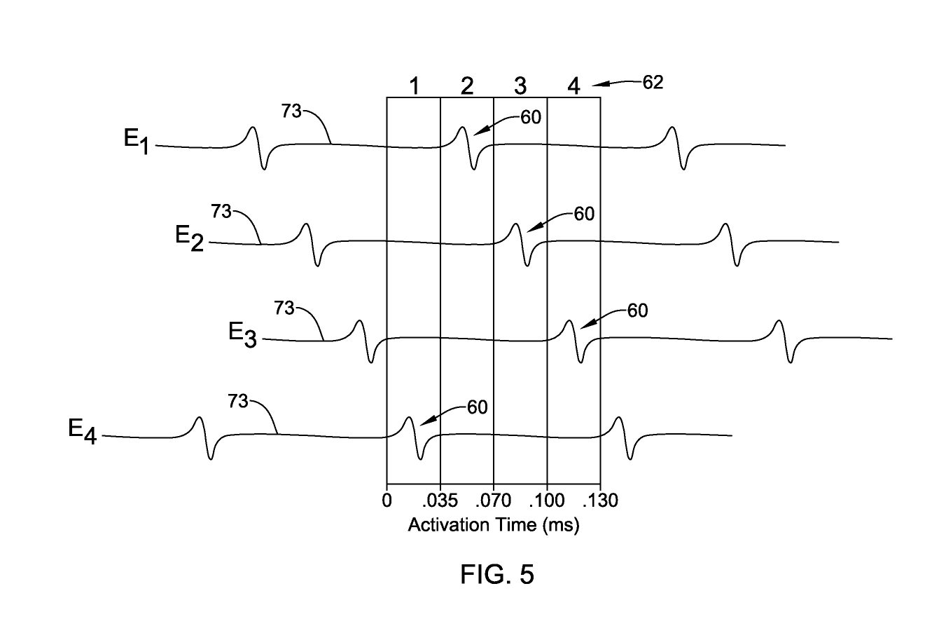

FIG. 5 illustrates schematically how processing system 32 may begin to characterize the activation times as part of a data reduction process. Here a series of electrograms 73 are shown. Each electrogram 73 may represent the change in electrical potential sensed by an example electrode. For convenience, four electrograms 73 are shown and are labeled as being representative of an example electrode E1, an example electrode E2, an example electrode E3, and an example electrode E4. These electrodes may correspond to electrodes 24 of multiple electrode structure 20. In practice, electrograms 73 may be formed for all of electrodes 24 of multiple electrode structure 20.

A fiducial point 60 may be identified by processing system 32 for each electrogram 73. As suggested herein, fiducial point 60 may correspond to a predetermined threshold change in electrical potential over a threshold time (e.g. the slope of the curve on the electrogram). Accordingly, fiducial points 60 may be used to define the activation time for each of the electrodes. For the purposes of this disclosure, the time at which fiducial point 60 occurs relative to a reference time (the reference time may be the time a reference electrode senses a cellular activation wavefront and, for convenience, is set to 0 ms) corresponds to the activation time for a given electrode. The various times that are determined as the activation times for each of the electrodes may be sorted or “bucketed” into different rank categories 62. Each rank category 62 may correspond to a range of activation times or discrete time intervals.

In some embodiments, generating discrete time intervals may include creating a histogram of the activation times of the subset of signals. A histogram is one example of a statistical methodology for analyzing a given set of data. In practice, processing system 32 may be configured to analyze and compute sensed data such as a histogram or visual representation. The actual data may or may not be actually formed and/or displayed. Processing system 32 may utilize the Freedman-Diaconis rule to create discrete equal time intervals based on the cellular activation times of the subset of sensed signals. It is contemplated the other statistical rules may be utilized to compare activation times of the electrical signals. Further, it is contemplated that in some embodiments the discrete time intervals may not be equal.

By bucketing fiducial points 60 into discrete rank categories 62, the raw activation time data may be simplified into smaller groupings based on similarities. For example, rank categories 62 may take the form of integers that correspond to a particular range of activation times. As such, fiducial points 60 that occur within a particular range of activation times will be assigned the corresponding rank category 62. For example, electrode E1 displays fiducial point 60 occurring at a time falling within the time range corresponding to rank category 62 classified as “2”. In FIG. 5, four integers are used as rank categories 62. It can be appreciated that more or fewer integers may be utilized. While integers may be convenient for use as rank categories 62, other indicators may also be used including colors, letters, symbols, textures, patterns, or the like.

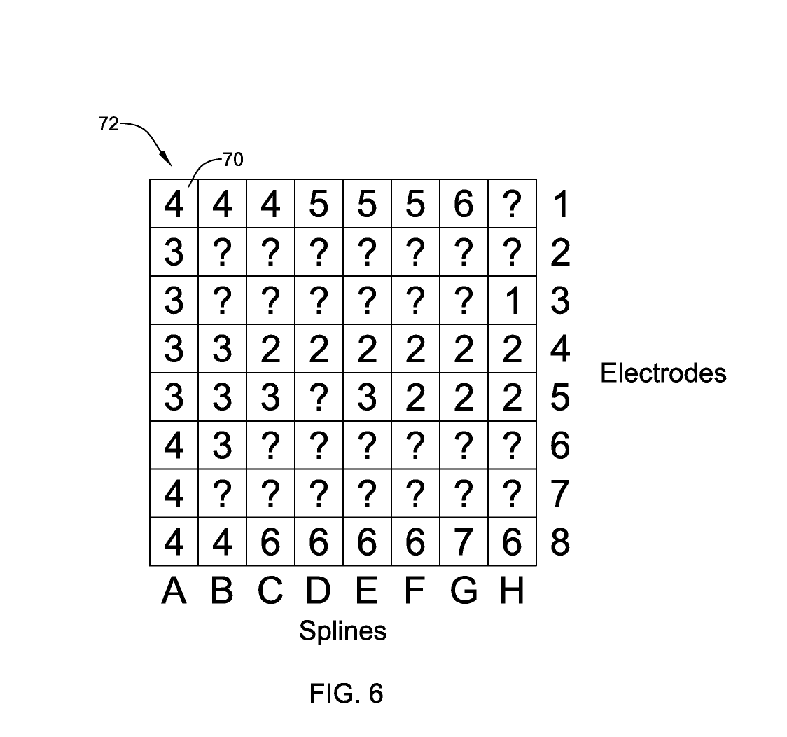

It can be appreciated that the data reduction process may be carried out (e.g., by processing system 32) for each electrode 24 of multiple electrode structure 20. For example, FIG. 6 illustrates a “data-reduced” activation map 72 where integers ranging from 1-7 are utilized as different rank categories 62 corresponding to the discrete ranges of activation times for electrodes 24. Space 70, which displayed an activation time of 0.101 ms in FIG. 4, now displays a rank category of “4”.

As stated above, in a normal functioning heart, electrical discharge of the myocardial cells may occur in a systematic, linear fashion. Therefore, detection of non-linear propagation of the cellular excitation wavefront may be indicative of cellular firing in an abnormal fashion. For example, cellular firing in a rotating pattern may indicate the presence of dominant rotors and/or divergent activation patterns. Further, because the presence of the abnormal cellular firing may occur over localized target tissue regions, it is possible that electrical activity may change form, strength or direction when propagating around, within, among or adjacent to diseased or abnormal cellular tissue. Identification of these localized areas of diseased or abnormal tissue may provide a clinician with a location for which to perform a diagnostic procedure. For example, identification of an area consisting of reentrant or rotor currents may be indicative of an area of diseased or abnormal cellular tissue. The diseased or abnormal cellular tissue may then be targeted for an ablative procedure. The activation time mapping grid 72 may be used to identify areas of circular, adherent, rotor or other abnormal cellular excitation wavefront propagation.

In order to maximize the utility of activation time map 72, it may be desirable to populate unknown activation time identifiers. Therefore, in some embodiments it may be desirable to choose and/or assign activation times (and corresponding rank categories 62) for missing signal data and populate and/or fill in the activation time map 72 accordingly.

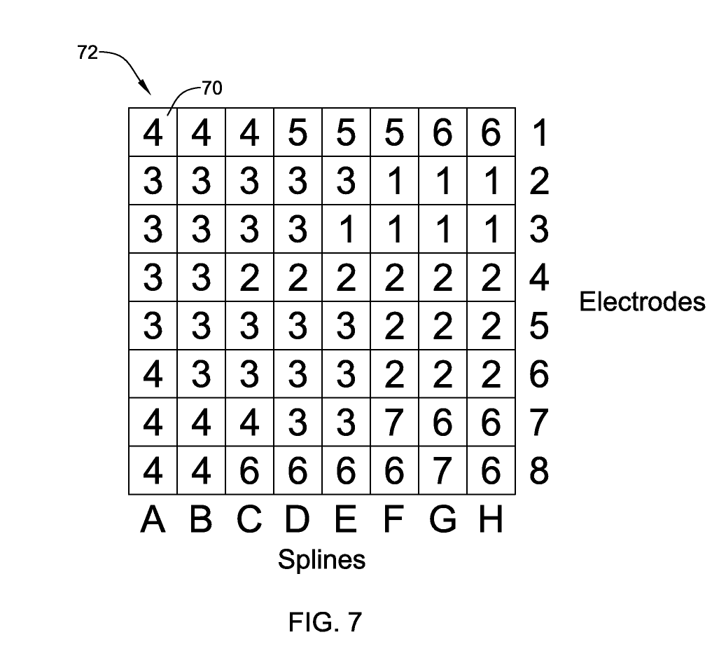

One method to choose and/or assign rank categories 62 and, thereby, fill in missing electrode data is to identify the physical position of all electrodes 24 in three-dimensional space, determine the distance between electrodes 24, and choose and/or assign activation times and corresponding activation time identifiers based on the physical distance between electrodes 24. In one embodiment, the physical distance between electrodes 24 may be determined by calculating the “linear” or “Euclidean” distance between electrodes 24. In non-curved space, it is generally understood that the shortest distance between two points is a straight line. Therefore, in some embodiments it may be desirable to choose and/or assign activation times and activation time identifiers for missing signal data by determining the linear, or straight-line, distance between an electrode 24 having missing data and its nearest neighboring electrode 24 exhibiting acceptable signal data. The activation times and activation time identifier of the closest neighboring electrode 24 exhibiting acceptable signal data may be “adopted” and/or assigned to electrode 24 having missing data. FIG. 7 illustrates a schematic activation map 72 that has been populated and/or filled in based on chosen and/or assigned missing activation time data and activation time identifiers.

In another embodiment, the physical distance between electrodes 24 may be determined by calculating the “geodesic” distance between electrodes 24. Geodesic distances may be understood to be the shortest distance between two points in curved space. It is generally understood that the anatomical shape of the interior walls of the heart are curved spaces. Further, because multiple electrode structure 20 may be configured to match the anatomical space in which it is deployed (e.g. heart chamber), electrodes 24 disposed on multiple electrode structure 20 may similarly be deployed along curved spaces. Therefore, the geodesic distance may be the shortest path between electrodes 24 along a curved path of cellular tissue.

An example method for calculating the geodesic distance may include creating a mesh (e.g., a coarse triangular mesh) between electrodes 24. The coarse triangular mesh may then be refined and/or upsampled. The refined mesh may then be utilized to calculate the geodesic distance between electrodes. After the geodesic distances are determined between electrodes 24, it may be desirable to choose and/or assign activation times and activation time identifiers for missing signal data by comparing the geodesic distance between an electrode 24 having missing data and its nearest neighboring electrode 24 exhibiting acceptable signal data. The activation times and activation time identifier of the closest neighboring electrode 24 exhibiting acceptable signal data may be “adopted” and/or assigned to electrode 24 having missing data.

While a triangular mesh may be useful, other geometries may be utilized. For example, the mesh may include other geometric shapes and/or configurations such as a polygon (e.g., having 4, 5, 6, 7, 8, 9, 10, or more sides), regular polygons, irregular polygons, etc.

In another embodiment, it may be desirable to choose and/or assign activation times and activation time identifiers for missing signal data by determining the “natural” distance between an electrode 24 having missing data and its nearest neighboring electrode 24 exhibiting acceptable signal data. The natural distance may be defined as distance between electrodes 24 lying on the same spline 44 as one another. Therefore, determining the natural distance between an electrode having missing data and the an electrode exhibiting acceptable data may include determining the distance between electrodes 24 exhibiting acceptable data which are lying on the same spline 44 as an electrode 24 having missing data. The activation times and activation time identifier of the closest neighboring electrode 24 exhibiting acceptable signal data (lying on the same spline 44) may be “adopted” and/or assigned to electrode 24 having missing data.

In another embodiment, the distance between electrodes 24 may be determined by using a distance kernel. The distance kernel may incorporate “thresholding” to further refine, compute, calculate and/or determine the distance between electrodes 24. A distance kernel may be used as a “weighting” or “probability” function that determines a more accurate estimation of the distance between electrodes 24. For example, based on the proximity of an electrode 24 having missing data relative to two different electrodes 24 exhibiting acceptable data, the distance kernel may, based on a statistical confidence and/or probability function, “weigh” the confidence and/or contribution from one of the electrodes 24 exhibiting acceptable data more than the other electrode 24 exhibiting acceptable data. It is contemplated that any number of electrodes exhibiting acceptable data may be utilized and/or incorporated into the distance kernel approximation. The statistical confidence and or probability functions employed by the distance kernel may incorporate Gaussian distributions to further refine the estimation of the distance between electrodes 24. Based on the calculation and/or estimation of the distance kernel, the activation times and activation time identifier of the closest neighboring electrode 24 exhibiting acceptable signal data may be “adopted” and/or assigned to electrode 24 having missing data.

In another embodiment, one or more methods stated above (e.g., linear, natural, geodesic, and/or distance kernel) to determine electrode distance may be incorporated, included, used, utilized, and/or integrated into a processing system 32. Processing system 32 may be configured such that one or more methods stated above (e.g., linear, natural, geodesic, and/or distance kernel) to determine electrode distance may be implemented to populate and/or fill in electrodes 24 having missing data on activation map 72. Further, processing system 32 may incorporate an “iterative” process to assess, populate and/or fill in electrodes 24 having missing data on activation map 72. The iterative process may cycle through the process of determining an electrode 24 that has missing data, utilizing a methodology (e.g., linear, natural, geodesic, and/or distance kernel) to determine electrode distance, selecting an electrode 24 having an acceptable activation time, and populating and/or filling in the corresponding activation time indicators on activation map 72. The processing system 32 may integrate and/or employ a feedback loop in the iterative process. For example, the processing system 32 may integrate and/or employ a feedback loop when choosing and/or assigning activation times and corresponding activation time indicators and populating and/or filling in activation times and corresponding activation time indicators in activation map 72. A feedback loop may be designed to permit an operator (e.g. physician, clinician) the ability to select the number of iterations processing system 32 will implement to populate activation map 72. For example, a user (e.g. physician, clinician) may be able to input the number of iterations that processing system 32 will implement in populating activation map 72. It is further contemplated that processing system 32 may be include a preset maximum number of iterations that it will implement in populating activation map 72.

In another embodiment, processing system 32 may integrate and/or employ a combination of the stated methodologies (e.g., linear, natural, geodesic, and/or distance kernel) to determine electrode distance. A feedback loop may be incorporated within any of the stated methodologies (e.g., linear, natural, geodesic, and/or distance kernel) to determine electrode distance.

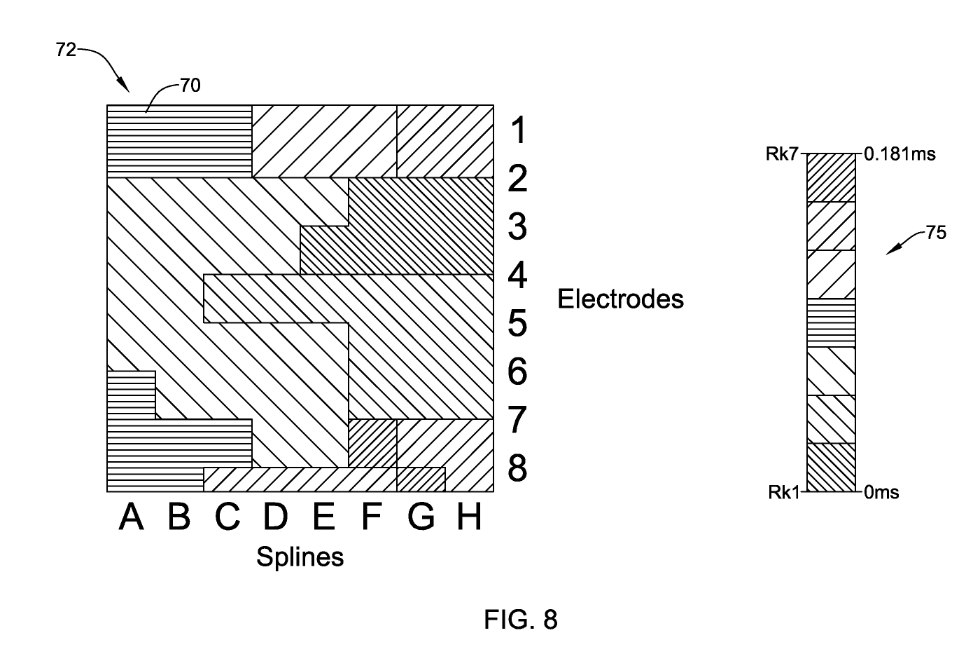

In at least some embodiments, rank categories 62 may be output or displayed as colors where each unique rank category 62 may be assigned a unique, differentiating color. For example, rank category 62 of “1” may be assigned the color red and rank category 62 of “2” may be assigned the color orange. It is contemplated that a variety of color combinations may be included in generating the color-based activation time map. Further, the color map may be displayed on a display. Additionally, the color map may help a clinician identify the direction of propagation of cellular firing. In some embodiments, each unique rank category 62 may be assigned a unique, differentiating pattern or texture in activation map 72 as shown in FIG. 8. Each pattern/texture may correspond to a particular rank category 62 and the corresponding range of activation times as schematically illustrated at a key 75. In at least some embodiments, the patterned/textured activation map 72 and key 75 may both be shown on a display.

In at least some of the embodiments described above the disclosed methods assume analysis of sensed, collected, measured and transmitted electrical cellular data occurring during a single heartbeat and/or cardiac pulse. However, it is contemplated that any of the disclosed methods may be implemented across multiple beats or cardiac pacing time intervals. Further, data collected over multiple heart beats may be analyzed using statistical methodologies and applied to the disclosed methods. For example, activation times may be collected over a series of heart beats and/or pulses. A statistical distribution of the collected activation times may be calculated, analyzed and incorporated into disclosed methods.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A medical device, comprising:
a catheter shaft with a plurality of electrodes coupled thereto; and
a processor coupled to the catheter shaft, wherein the processor is capable of:
collecting a set of signals from at least some of the plurality of electrodes;
generating a data set from the set of signals;
performing a data reduction process on the data set; and
generating a visual representation of the data set.

2. The medical device of claim 1, wherein collecting the set of signals includes sensing a change in electrical potential by any one of the plurality of electrodes.

3. The medical device of claim 2, further comprising identifying a threshold value corresponding to a minimum change in electrical potential by any one of the plurality of electrodes and wherein collecting the set of signals includes collecting only those signals that are above the threshold value.

4. The medical device of claim 1, wherein collecting the set of signals includes determining an activation time at one or more of the plurality of electrodes.

5. The medical device of claim 4, wherein determining the activation time includes identifying a fiducial point corresponding to a change in electrical potential and determining a time latency between a reference point and the fiducial point.

6. The medical device of claim 4, wherein performing the data reduction process on the data set includes assigning a rank category to the activation times, and wherein each rank category corresponds to a discrete activation time interval.

7. The medical device of claim 1, wherein generating a visual representation includes creating an activation map and wherein the activation map includes a grid displaying a plurality of activation times, a plurality of rank categories, or both.

8. The medical device of claim 7, wherein the activation map includes activation times from only some of the plurality of electrodes and includes one or more missing activation times.

9. The medical device of claim 8, further comprising assigning activation times to the one or more missing activation times.

10. The medical device of claim 9, wherein assigning activation times to the one or more missing activation times includes adopting the value of at least one activation time.

11. The medical device of claim 7, wherein the activation map includes rank categories from only some of the plurality of electrodes and includes one or more missing rank categories.

12. The medical device of claim 11, further comprising assigning rank categories to the one or more missing rank categories.

13. The medical device of claim 12, wherein assigning rank categories to the one or more missing rank categories includes adopting the value of at least one rank category.

14. The medical device of claim 7, wherein the activation map further comprises a plurality of color indicators, and wherein the color indicators represent activation times, rank categories, or both.

15. The medical device of claim 14, wherein the visual representation includes a key correlating the color indicators to the activation times, rank categories or both.

16. The medical device of claim 15, wherein the visual representation is displayed on a display.

17. A medical device for mapping a cardiac chamber, comprising:
a catheter shaft with a plurality of electrodes coupled thereto;
a processor, wherein the processor is capable of:
collecting a set of signals from at least some of the plurality of electrodes;
generating a data set from at least one of the set of signals, wherein the data set includes at least one known data point and one or more unknown data points;
performing a data reduction process on the known data point; and
assigning a value to at least one of the unknown data points.

18. The medical device of claim 17, wherein generating the data set includes determining an activation time at one or more of the plurality of electrodes with the at least one known data point and wherein performing the data reduction process on the data set includes assigning a rank category to the activation times.

19. The medical device of claim 18, wherein each rank category corresponds to a discrete activation time interval and wherein assigning a value to at least one of the unknown data points includes adopting the value of at least one activation time.

20. A method of mapping the electrical activity of the heart, the method comprising:
advancing a catheter shaft with a plurality of electrodes coupled thereto to a chamber of the heart, wherein the catheter shaft is coupled to a processor, and wherein the processor is configured to:
collect a set of signals from at least some of the plurality of electrodes;
generate a data set from the set of signals;
perform a data reduction process on the data set; and
generate a visual representation of the data set.
